(19) United States
(12) Patent Application Publication
(10) Pub. No.: US 2020/0009104 A1
(43) Pub. Date: Jan. 9, 2020

(54) COMBINATION BETWEEN TRIFLURIDINE/TIPIRACIL HYDROCHLORIDE, AN ANTITUMOR PLATINIUM COMPLEX, AND AN IMMUNE CHECKPOINT MODULATOR

(71) Applicant: LES LABORATOIRES SERVIER, SURESNES CEDEX (FR)

(72) Inventors: Jean-Pierre ABASTADO, Paris (FR); Nadia AMELLAL, Vincennes (FR); Alain BRUNO, Levallois (FR); Michael BURBRIDGE, Verneuil Sur Seine (FR); Valérie CATTAN, Paris (FR); Catherine LEGER, Meudon (FR)

(21) Appl. No.: 16/490,656

(22) PCT Filed: Mar. 16, 2018
(86) PCT No.: PCT/EP2018/056632

§ 371 Date: Sep. 3, 2019

(30) Foreign Application Priority Data
Mar. 17, 2017 (EP) 17161630.3

(51) Int. Cl. A61K 31/282; C07K 16/28; A61K 31/7072; A61K 31/513; A61K 9/00; A61P 35/00

(52) U.S. Cl. CPC A61K 31/282; C07K 16/2818; A61K 31/7072; A61P 35/00; A61K 9/0019; A61K 9/0053; A61K 31/513

(57) ABSTRACT

A combination comprising:

an FTD-TPI drug,

an anti-tumor platinum complex,

and an immune checkpoint modulator. Medicinal products containing the same which are useful in treating cancer.

The present invention relates to an anti-tumor agent comprising a combination of i) trifluridine and tipiracil hydrochloride, ii) an anti-tumor platinum complex and iii) an immune chekpoint modulator. Trifluridine (another name: α,α,α-trifluorothymidine; hereinafter also called “FTD”) causes function inhibition of DNA by being incorporated into DNA of a tumor cell, and exhibits anti-tumor effects. Meanwhile, tipiracil hydrochloride (chemical name: 5-chloro-6-[(2-iminopyrrolidin-1-yl)methyl]-pyrimidine-2,4(1H,3H)-dione hydrochloride; hereinafter also called “TPI”) has a thymidine phosphorylase inhibitory effect. It is known that TPI prevents in vivo degradation of FTD by thymidine phosphorylase, thus enhancing the anti-tumor effect of FTD (Investigational New Drugs, 26(5), 445-454, 2008). At the present time, an anti-tumor agent comprising FTD and TPI at a molar ratio of 1:0.5 (hereinafter also called “FTD-TPI drug” or “TAS-102”) has been developed as a therapeutic agent of solid cancers and is approved in Japan as a therapeutic agent for unresectable advanced or recurrent colorectal cancer, and in United States and Europe under tradename Lonsurf® (EMA/CHMP/130102/2016) as a therapeutic agent for metastatic colorectal cancer which has been previously treated with available therapies including fluoropyrimidin, oxaliplatin and irinotecan based chemotherapies, anti-VEGF (Vascular Endothelial Growth Factor) agents and anti-EGFR (Epidermal Growth Factor receptor) agents.

FTD-TPI drug and its use has been described for example in patent EP763529.

In order to enhance the anti-tumor effect of the FTD-TPI drug, combination therapies have been studied in preclinical experiments, and the studies have suggested positive combination effects with irinotecan, oxaliplatin, docetaxel, or the likes (European Journal of Cancer, 43(1), 175-183, 2007; British Journal of Cancer, 96(2), 231-240, 2007; Cancer Science, 99(11), 2302-2308, 2008).

On a clinical aspects, a huge number of combinations have been envisaged and evaluated, particularly in metastatic colorectal cancer treatment. Second line intensive therapy is normally proposed for patients with good performance status and adequate organ function. Combination second line therapies with oxaliplatin and irinotecan are known to be superior to best supportive care, but outcomes remains poor with a median progression-free survival ranging from 5.7 to 7.4 months and a median overall survival ranging from 12.5 to 14.5 months. As a consequence, there is still a need for new combinations providing better activity and better outcomes.

Anti-tumor platinum complexes are metal complex compounds containing platinum as the central metal, and inhibit DNA replication by binding to DNA, thus exerting anti-tumor effects. Platinum complexes as anti-tumor agents have been studied for a long time, and cisplatin, carboplatin, oxaliplatin, and the likes are clinically used against a wide variety of cancer types (Annales Pharmaceutiques Françaises, 69(6), 286-295, 2011). Combination use of anti-tumor platinum complexes with various anti-tumor agents has also been studied. In particular, combination use with an antimetabolite such as 5-fluorouracil is widely adopted.

More particularly, it has been shown that combination of FTD-TPI drug with oxaliplatin in murine microsatellite stable or mismatch repair proficiency MSS/pMMR colorectal cancer cell line CT26 induced immunogenic cell death in a higher level than oxaliplatin or FTD-TPI drug taken alone. This type of cell death is characterized by a compendium of subtle biochemical changes in the properties of the plasma membrane as well as in the microenvironment of dying cancer cells (EMBO Journal, 2012, 31(5), 1055-1057; Nature Reviews Immunology, 2009, 9(5), 353-363). These changes include:

the pre-apoptotic exposure of calreticulin (CRT) on the cell surface, which facilitates the engulfment of portions of the dying cells by antigen-presenting cells (Cell, 2005, 123(2), 321-334);

the post-apoptotic release of high mobility group box 1 (HMGB1) from the nucleus which stimulates antigen presentation (Nature Medicine, 2007, 13(9), 1050-1059);

the release of ATP essential for cell death to be perceived as immunogenic.

The induction of immunogenic cell death is of particular interest in stimulating a therapeutic immune response. Induction of immunogenic cell death combined with reactivation of a proficient immune response with checkpoint modulator against malignant cells should be associated with improved disease outcomes. Checkpoint therapy is a promising approach against cancer and consists of targeting immune checkpoints such as programmed cell death protein 1 (PD-1), programmed cell death 1 ligand 1 (PD-L1) and cytotoxic T lymphocyte antigen 4 (CTLA4). Such approaches have achieved noteworthy benefit in multiple cancers by blocking immunoinhibitory signals and enabling patients to produce an effective antitumour response. Inhibitors of CTLA4, PD-1 or PD-L1 administered as single agents have resulted in durable tumour regression in some patients, and combinations of PD-1 and CTLA4 inhibitors may enhance antitumour benefit.

Anti-PD-1 immune checkpoint inhibitors have shown encouraging results in patients with microsatellite instable or mismatch repair deficiency MSI/dMMR colorectal cancer (The New England Journal of Medicine, 2015, 372(26), 2509-2520) but MSI/dMMR represents only 5% of patients in the metastatic setting (Journal of the National Cancer Institute, 2013, 105(15), 1151-1156). The activity of immune checkpoint inhibitor in MSI/dMMR patients can be explained by higher mutational load in MSI/dMMR tumors that creates many tumor-specific neoantigens. In contrast, the majority of patients with MSS/pMMR colorectal cancer do not respond to anti-PD-1 mono-therapy (The New England Journal of Medicine, 2015, 372(26), 2509-2520) and it is imperative to study combinations involving immunotherapy drugs and chemotherapies that are able to promote tumor immunity.

The present invention proposes a new combination based first on the mechanism of increasing tumor immunogenicity with an ad hoc treatment such as FTD-TPI drug and platinum complexe in order to enhance then tumor response to an immune checkpoint modulator. Such new combination treatment would include MSS/dMMR tumor cancers. Especially, the applicant has shown that in vivo combination of FTD-TPI drug with an anti-tumor platinum complex and an immune checkpoint modulator on a CT26 MSS/dMMR colorectal carcinoma bearing mice showed higher survival compared to FTD-TPI drug combined to the immune checkpoint modulator or the doublet chemotherapy FTD-TPI drug and anti-tumor platinum complex, with statistical significance.

According to a first aspect of the invention, there is provided a combination comprising:

the FTD-TPI drug,

an anti-tumor platinum complex,

and an immune checkpoint modulator, for concomitant or sequential therapeutic use.

By concomitant therapeutic use, within the meaning of the present invention is meant in the present application an administration of the three components of the combination at the same time or at substantially the same time, i.e. within 24 hours, the administration route being identical or different.

By sequential therapeutic use, within the meaning of the present invention is meant in the present application an administration of at least two components of the combination at different times, the administration route being identical or different. The administration at different time will be preferably from 24 hours to 14 days later, and more preferably from 7 to 14 days later for at least one of the components of the combination.

In another embodiment, the invention provides a combination as described herein, for use in the treatment of cancer.

More particularly, combination of the invention will be useful for the treatment of esophageal, gastric, liver, gallbladder/bile duct, stomach, liver, pancreatic, colorectal, ovarian, uterin, head and neck, thyroid, lung, breast, cervical, bladder, testicular and prostate cancers, sarcomas, skin cancer, malignant lymphoma, acute leukemia, and brain tumors.

Advantageously, the combination of the invention will be useful for the treatment of colorectal cancer, and more preferably metastatic colorectal cancer.

Alternatively, the combination of the invention will be useful for the treatment of gastric cancer.

In another embodiment, the invention provides a medicament containing, separately or together:

the FTD-TPI drug,

an anti-tumor platinum complex,

and an immune checkpoint modulator, for concomitant or sequential administration, and wherein each component are provided in effective amounts for the treatment of cancer.

“Combination” refers to either a fixed dose combination in one unit dosage form (e.g., capsule, tablet, or sachet), non-fixed dose combination, or a kit of parts for the combined administration where components may be administered independently at the same time or separately within time intervals, especially where these time intervals allow that the combination partners show a cooperative effect.

The term “fixed dose combination” means that the active ingredients are both administered to a patient simultaneously in the form of a single entity or dosage.

The term “non-fixed dose combination” means that the active ingredients are administered to a patient as separate entities either concomitantly or sequentially, with no specific time limits, wherein such administration provides therapeutically effective levels of the active ingredients in the body of the patient.

“Cancer” means a class of disease in which a group of cells display uncontrolled growth.

Cancer types include haematological cancer (lymphoma and leukemia) and solid tumors including carcinoma, sarcoma, or blastoma. In particular “cancer” refers to esophageal, gastric, liver, gallbladder/bile duct, stomach, liver, cholecystic-cystic duct, pancreatic, colorectal, ovarian, head and neck, lung, breast, cervical, bladder, testicular and prostate cancers, bone sarcoma, skin cancer, malignant lymphoma, acute leukemia, and brain tumors, chronic leukemia, meduloblastoma, retinoblastoma, neuroblastoma, Wilm's tumor, Hodgkin's disease, multiple myeloma, plasmocytoma, thymoma, basal cell cancer, squamous cancer, Ewing's tumor, thyroid gland cancer, ovarian cancer, salivary gland cancer, teratoma, malignant melanoma, glioma, renal cell cancer, osteosarcoma. Colorectal cancer, and preferentially metastatic colorectal cancer, and gastric cancer are particularly preferred.

“Treatment cycle” means a period of time to receive treatment according to a determined administration schedule after which the efficacy of the treatment is assessed by evaluating the tumour response.

The FTD-TPI drug of the invention relates to a combination containing FTD and TPI at a molar ratio of 1:0.5. The dosage regimen is usually as follows: the combination drug is orally administered at a usual dose of 20 to 80 mg/m²/day in terms of FTD in two divided portions per day for five consecutive days, and then a 2-days rest period is taken. This cycle is repeated twice, and then a 14-days rest period is taken. Alternatively, the dosage regimen for the FTD-TPI drug is as follows: FTD-TPI drug is administrated at a dose of 20 to 80 mg/m²/day in terms of FTD in two divided portions per day for five consecutive days, and then a 9-days rest period is taken, resulting in a 14-days cycle of treatment.

The definition of the “anti-tumor platinum complex” in the present invention is part of common general technical knowledge, and the anti-tumor platinum complex may be any compound that has a platinum complex as the central metal and has anti-tumor activities. The anti-tumor platinum complex is specifically exemplified by cisplatin, carboplatin and oxaliplatin. Of them, particularly preferred is oxaliplatin. The anti-tumor platinum complex of the present invention includes drug delivery system (DDS) preparations containing the anti-tumor platinum complex as an active ingredient (for example, micellar cisplatin and liposomal oxaliplatin).

More particularly, oxaliplatin (chemical name: [(1R,2R)-cyclohexane-1,2-diamine](ethane dioato-O,O′)platinum(II)) is a known compound commercialized as Eloxatin®.

The recommended dose for oxaliplatin in treatment of metastatic colorectal cancer is 60 to 90 mg/m², and more preferably 65 to 85 mg/m² intravenously repeated every 2 weeks until disease progression or unacceptable toxicity.

The immune checkpoint modulator of the invention is preferably a PD-1 pathway antagonist, an ICOS pathway antagonist, a CTL-4 pathway antagonist, a CD28 pathway antagonist or a combination thereof. More preferably, the immunomodulator of the invention is an anti-PD-1 antibody, an anti-PD-L1 antibody, or a combination thereof. The preferred immunomodulators of the invention are nivolumab, pembrolizumab, pidilizumab, atezolizumab, durvalumab and avelumab. Most preferred immunomodulators of the invention are nivolumab and pembrolizumab, and even more preferably nivolumab. The recommended dose for nivolumab (Opdivo®) is 3 mg/kg administered intravenously over 60 minutes every 2 weeks. Pembrolizumab (Keytruda®) is commonly administered as an intravenous infusiona at 2 mg/kg over 30 minutes every 3 weeks.

The compounds of the combination can be administered in a sequential or a concomitant way. By sequential way it is understood that at least two components of the combination are administered at different, times. A sequential preferred way is wherein at least one compound of the combination will be initiated after the two others, preferentially from 24 hours to 14 days later, and more preferably from 7 to 14 days later.

By concomitant way it will be understood that the three components of the combination are initiated within 24 hours. In the combination of the present invention, each component will be administered at a sequential or concomitant way, at a dose that is preferably 50 to 100% of the recommended dose for each when administered alone.

More particularly, the combination of the present invention will be administered in a concomitant way.

An advantageous alternative will be an administration in a sequential way. In that case, the administration of at least one of the components of the combination will be initiated after the others, preferentially 24 hours to 14 days later, and more preferably from 7 to 14 days later. More preferably, in a sequential way, the component initiated later will be the immune checkpoint modulator.

More preferably, a 14 days treatment cycle will be envisaged for FTD-TPI drug and the anti-tumor platinum complex, and the immune checkpoint inhibitor will be administered as recommended over 2 to 3 weeks depending on the treatment. In case of an immune checkpoint inhibitor to be administered every 3 weeks, the treatment cycle to consider will be a six weeks treatment.

Advantageously, the 14 days treatment cycle will be a concomitant way and will include:

the administration of FTD-TPI drug orally bid (twice a day) from Day 1 through Day 5, followed by a recovery period of 9 days on Day 6 through Day 14;

the administration on Day 1 of the antitumor platinum complex; and

the administration on Day 1 of the immune checkpoint inhibitor.

More advantageously, the 14 days treatment cycle will be a concomitant way and will include:

the administrations of FTD-TPI drug orally bid (twice a day) at a dose of 25, 30 or 35 mg/m²/dose in terms of FTD within 1 hour after completion of morning and evening meals, from Day 1 to Day 5, followed by a recovery period of 9 days on Day 6 through Day 14;

the administration on Day 1 of the recommended dose of the antitumor platinum complex intravenously, the start of the infusion being concomitant with the morning administration of FTD-TPI drug at Day 1; and

the administration of the recommended dose of the immune checkpoint modulator intravenously, the start of the infusion being concomitant with the morning administration of FTD-TPI drug at Day 1.

Even more preferably, the 14 days treatment cycle will be a concomitant way and will include:

the administrations of FTD-TPI drug orally bid (twice a day) at a dose of 25, 30 or 35 mg/m²/dose in terms of FTD within 1 hour after completion of morning and evening meals, from Day 1 to Day 5, followed by a recovery period of 9 days on Day 6 through Day 14;

the administration on Day 1 of oxaliplatin intravenously as 2-hours infusion at 85 or 65 mg/m², the start of the infusion being concomitant with the morning administration of FTD-TPI drug at Day 1; and

the administration of nivolumab at a dose of 3 mg/kg intravenously, the start of the infusion being concomitant with the morning administration of FTD-TPI drug at Day 1.

In the 14 days concomitant treatment described above, as an alternative, one of the components of the combination can be initiated after the two others, leading to a sequential way of administration. More particularly, the immune checkpoint immunomodulator can be initiated after the administration of both platinum complex and FTD-TPI drug. Preferentially the immune checkpoint immunomodulator treatment can be initiated 24 hours to 14 days later, and more preferably 7 to 14 days later than the two other components.

The 14 days treatment will be repeated as long as the treatment combination will benefit to the patients. One of the components of the combination can be stopped before the two others in case of toxicity related.

Among the pharmaceutical compositions according to the invention there may be mentioned more especially those that are suitable for administration by the oral, parenteral, intramuscular and intravenous, per- or trans-cutaneous, nasal, rectal, perlingual, ocular or respiratory route and more specifically tablets, dragées, sublingual tablets, gelatin capsules, glossettes, capsules, lozenges, injectable preparations, aerosols, eye or nasal drops, suppositories, creams, ointments, dermal gels, etc.

In addition to the active principles, the pharmaceutical compositions according to the invention comprise one or more excipients or carriers chosen from diluents, lubricants, binders, disintegrators, stabilisers, preservatives, absorbents, colourings, sweeteners, flavourings, etc.

Examples which may be mentioned, without implying any limitation, include:

for the diluents: lactose, dextrose, sucrose, mannitol, sorbitol, cellulose, glycerin;

for the lubricants: silica, talc, stearic acid and its magnesium and calcium salts, polyethylene glycol;

for the binders: aluminium and magnesium silicate, starch, gelatin, tragacanth, methylcellulose, sodium carboxymethylcellulose and polyvinylpyrrolidone;

for the disintegrators: agar, alginic acid and its sodium salt, effervescent mixtures.

The corresponding pharmaceutical compositions can permit the immediate or delayed release of the active ingredients. Moreover, the compounds of the combination can be administered in the form of three separate pharmaceutical compositions, each comprising one of the active ingredients, or alternatively in the form of a single pharmaceutical composition in which the active ingredients are mixed.

The dosage used for FTD-TPI drug varies according to the body surface of the patient, the administration route, the nature of the cancer and of any associated treatments, and the observed toxicity. It will ranges from 20 to 80 mg/m²/day in terms of FTD divided in two to four portions per day.

The dose of the anti-tumor platinum complex will be equal to that used when it is administered on its own or less. By way of example, in the case of oxaliplatin, the dose administered is determined according to AUC (Area under the curve), and ranges from 30 to 100 mg/m².

The dose of the immunomodulator will be equal to that used when it is administered on its own or less. By way of example, in the case of nivolumab, the dose administered is from 1 to 20 mg/kg.

PHARMACEUTICAL COMPOSITIONS Lonsurf® Film-coated tablets containing 15 mg trifluridine and 6.14 mg tipiracil (as hydrochloride) or 20 mg trifluridine and 8.19 mg tipiracil (as hydrochloride) as active substances. Other ingredients are:

Tablet core: lactose monohydrate, pregelatinized maize starch, stearic acid

Film coating: Lonsurf 15 mg/6.14 mg film-coated tablets: hypromellose, macrogol (8000), titanium dioxide (E171), magnesium stearate Lonsurf 20 mg/8.19 mg film-coated tablet: Hypromellose, macrogol (8000), titanium dioxide (E171), iron oxide red (E172), magnesium stearate Printing ink: shellac, iron oxide red (E172), iron oxide yellow (E172), titanium dioxide (E171), indigo carmine aluminium lake (E132), carnauba wax, talc

Eloxatin® 5 mg/ml of oxaliplatin concentrate for solution for infusion Water for injections

Opdivo® 10 mg/ml of nivolumab concentrate for solution for infusion

Sodium citrate dihydrate Sodium chloride Mannitol (E421) Pentetic acid (diethylenetriaminepentaacetic acid) Polysorbate 80 Sodium hydroxide (for pH adjustment) Hydrochloric acid (for pH adjustment) Water for injections

Keytruda® 50 mg of pembrolizumab (lyophilized powder) for solution for infusion L-histidine L-histidine hydrochloride, monohydrate Saccharose Polysorbate-80 After reconstitution, 1 ml of solution contains 25 mg of pembrolizumab

PRECLINICAL STUDIES

### BRIEF DESCRIPTION OF THE FIGURES:

FIGS. 1a, 1b and 1c: Analysis of impact of FTD-TPI drug and oxaliplatin exposure on immunogenic cell death induction; calreticulin (CRT) exposure and ATP release (FIG. 1a), high-mobility group box 1 (HMGB1) release (FIG. 1b) and Eukaryotic initiation Factor 2 (EIF2-α) phosphorylation (FIG. 1c).

FIG. 2: Analysis of impact of FTD-TPI drug and oxaliplatin exposure on immunogenic cell death induction in xenograft mice. a) Cytoplasmic HMGB1; b) tumoral pEIF2α/EIF2α ratio.

FIG. 3: Analysis of impact of FTD-TPI drug and oxaliplatin exposure on CD8-T cell infiltration in tumor in vivo.

FIG. 4: Schedule of administration of FTD-TPI drug, oxaliplatin and anti-mouse PD-1 antibody.

FIG. 5: Body weight and tumor growth of the combination of FTD-TPI drug, oxaliplatin and anti-mouse PD-1 antibody in murine CT26 MSS/pMMR colorectal carcinoma bearing mice.

FIG. 6: Survival of the exposure to combination of FTD-TPI drug, oxaliplatin and anti-mouse PD-1 antibody in murine CT26 MSS/pMMR colorectal carcinoma bearing mice.

A) Impact of FTD-TPI drug and oxaliplatin on immunogenic cell death induction in tumor cells in vitro The objective of the study is to assess the potential of FTD-TPI drug (TAS-102) alone or in combination with oxaliplatin to induce immunogenic cell death in murine MSS/pMMR CT26 colorectal cancer cells in vitro. First an analysis of cellular response is monitored after TAS-102 exposure at a dose of 500, 50, 5, 0.5 or 0.05 μM in terms of FTD, with or without oxaliplatin at a dose of 500, 50, 5, 0.5 or 0.05 μM at different time points (24 h and 48 h). FTD-TPI drug and oxaliplatin combination ratio is 1:1. Drug response has been analysed by staining of adherent cells in 96 well plates (crystal violet).

Three doses were chosen to test the induction of cell death in 24 well plates at the time point “48 h” both by crystal violet staining and by flow cytometry (Annexin-V/7AAD (7-amino-actinomycin D) staining).

After these analyses the next immunogenic cell death (ICD) relevant markers were analysed:

plasmic membrane calreticulin (CRT) exposition, analysed by flow cytometry (48 h)

high-mobility group box 1 (HMGB1) secretion analysed by ELISA (Chondrex)

ATP secretion assayed by fluorimetry (Promega) ICD marker analysis has been performed with two different FTD-TPI drug concentrations in combination or not with oxaliplatin. Mitoxantrone is used as a positive control. Eukaryotic initiation Factor 2 (EIF2-α) expression and phosphorylation has been tested to confirm the results with an additional marker of the ICD induction. This confirmation is important to validate ICD induction in CT26 model used for in vivo experiment.

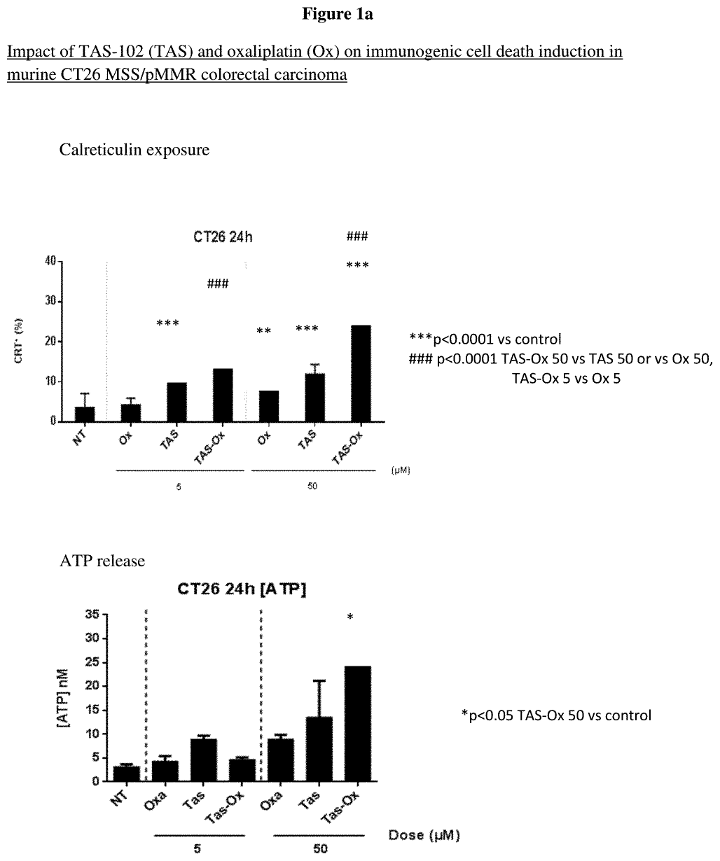

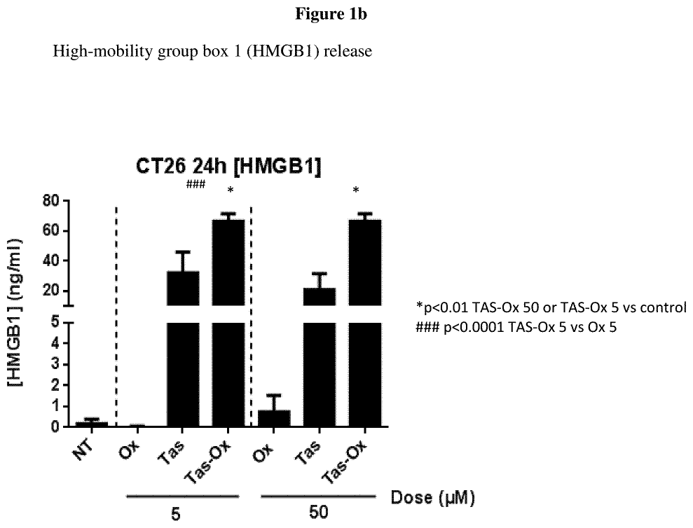

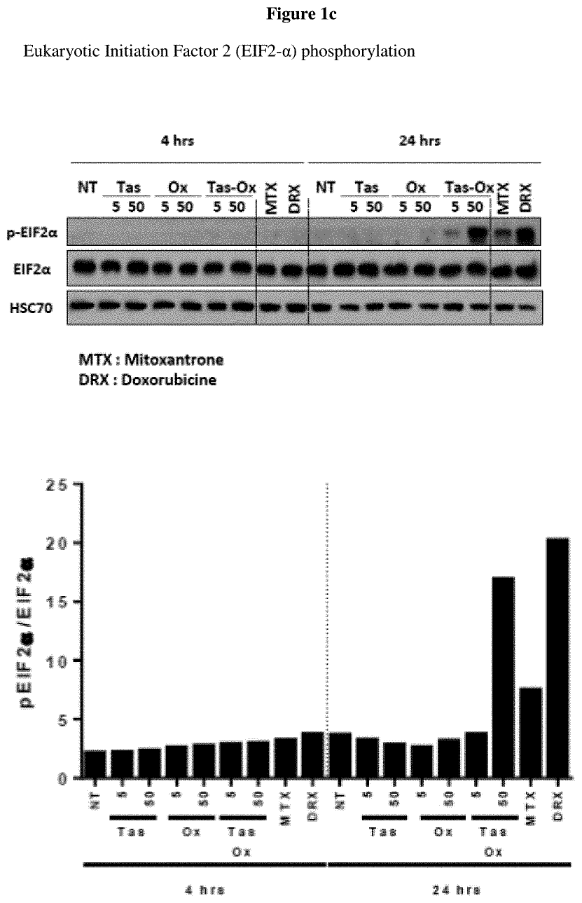

Treatment with FTD-TPI drug in combination with oxaliplatin in murine MSS/pMMR colorectal cancer cell lines CT26 resulted in emission of damage-associated molecular patterns such as cell surface exposure of calreticulin, exposure and phosphorylation of EIF2α, high-mobility group box 1 (HMGB1) and ATP release, characteristics of immune cell death, in a higher level than control (Mitoxantrone) or oxaliplatin alone or FTD-TPI drug alone (See FIGS. 1a, 1b, 1c).

B) Impact of FTD-TPI drug and oxaliplatin on immunogenic cell death induction in tumor in vivo

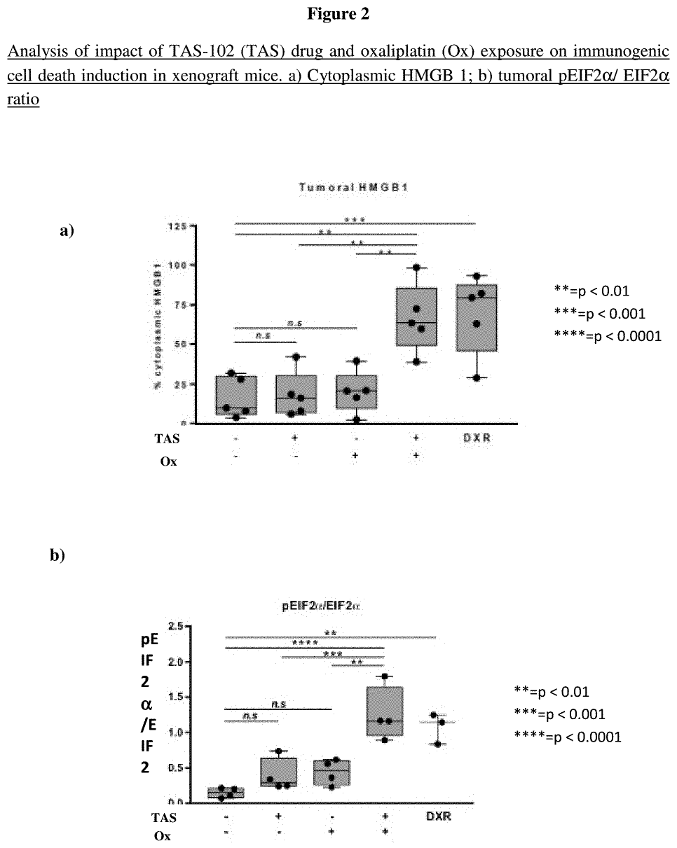

The objective of the study is to assess the potential of FTD-TPI drug (TAS-102) alone or in combination with oxaliplatin to induce immunogenic cell death (ICD) in vivo in MSS/pMMR CT26 colorectal cancer xenograft mice. CT26 tumor cells were injected into the right flank of Balb/c mice (1.10⁶cells). Ten days after tumor implantation, mice were randomized and received FTD/TPI (per os, 150 mg/kg/d) and/or oxaliplatin (ip, 6 mg/kg/w) for 3 days. Intratumoral injection of Doxorubicin (3 mg/kg/w) was used as positive control. Cytoplasmic HMGB1 in tumors, marker of ICD was assessed by immunochemistry and pEIF2α/EIF2α ratio by western blot 13 days after tumor implantation. The combination of FTD-TPI drug with oxaliplatin induced in vivo synergistic immunogenic cell death attested by cytoplasmic release of HMGB1 and phosphorylation of pEIF2α in tumor xenograft (See FIG. 2, p<0.01 versus either drug alone for HMGB1; p<0.01 versus oxaliplatin for pEIF2α; p<0.001 versus FTD-TPI for pEIF2α).

C) Impact of FTD-TPI drug and oxaliplatin on CD8-T cell infiltration and CD8-T cell functionality in tumor in vivo

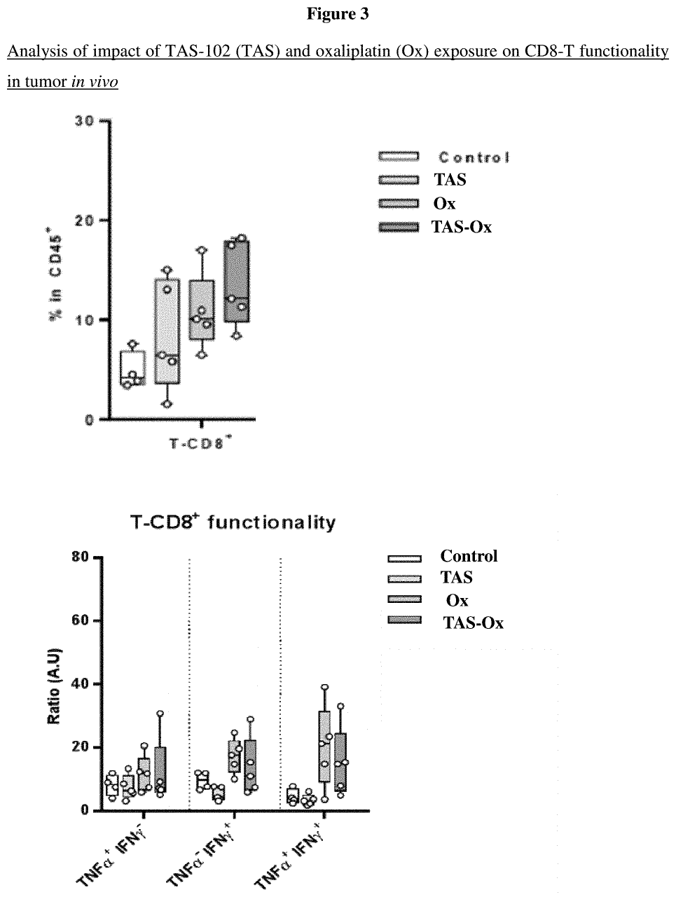

The objective of the study is to assess the potential of FTD-TPI drug (TAS-102) alone or in combination with oxaliplatin to induce in vivo CD8-T cell infiltration in MSS/pMMR CT26 colorectal cancer xenograft mice and assess the functionality of CD8-T cells assessed by TNFα and INFγ expression. CT26 tumor cells were injected into the right flank of Balb/c mice (1.10⁶cells). Ten days after tumor implantation, mice were randomized and received FTD/TPI (per os, 150 mg/kg/d) and/or oxaliplatin (ip, 6 mg/kg/w) for 4 days. Tumor CD8-T cells infiltrate analysis was perfomed by flow cytometer 18 days after tumor implantation. The combination of FTD-TPI drug with oxaliplatin induced in vivo significant CD8-T cells tumor infiltration compared to control mice (p<0.05). CD8-T cells infiltration following FTD-TPI and oxaliplatin treatment is associated with increased INFγ expression (See FIG. 3).

Overall results indicates that treatment with FTD-TPI and oxaliplatin is able to induce ICD that favours CD8-T cells infiltration and activation showing an adaptative immune response against tumor cells. A foundational principle of tumor immunology is that cancer cells can be eliminated by cytotoxic CD8-T cells (Schreiber et al., 2011, Science 331(6024), 1565-1570; Gajewski et al., 2013, Nat. Immunol., 14(10), 1014-1022; Schumacher and Schreiber, 2015, Science 348(6230), 69-74). These cells can be subject to various suppressive mechanisms including inhibitory immune checkpoint receptors expression. Immune checkpoint inhibitors prevent this immunosuppressive signal and allow tumor-specific T cells to remain activated and kill tumor cells. Tumors that lack antigen presentation or are devoid of T cells are significantly less likely to respond to Immune checkpoint inhibitors. By mediating anticancer immunity, FTD-TPI combined with oxaliplatin has the potential to expand the number of patients who could benefit from Immune checkpoint inhibitors.

D) Anti-tumour efficacy of FTD-TPI drug in combination with oxaliplatin and an anti-mouse PD-1 monoclonal antibody using murine colorectal carcinoma (CRC)-bearing mice

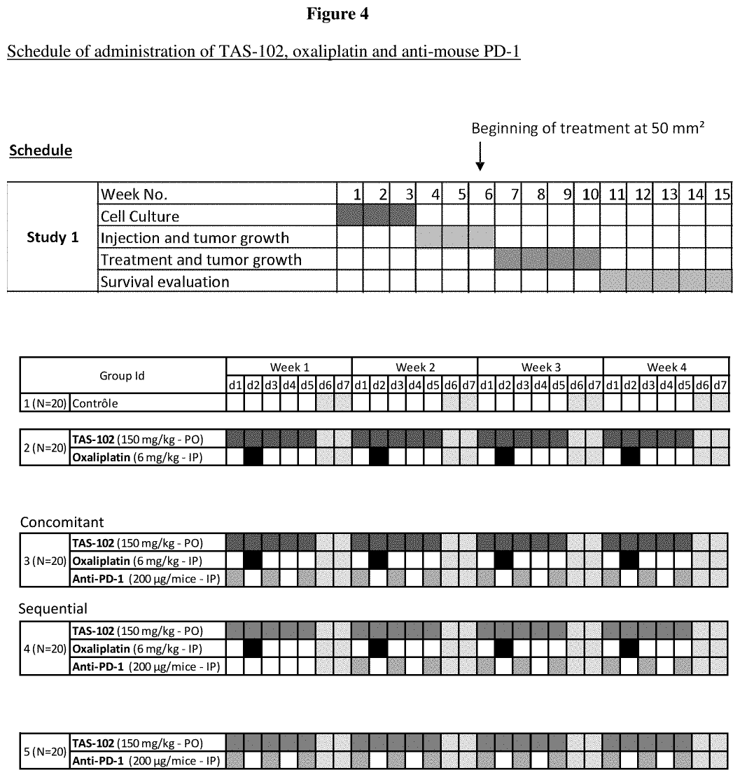

This study is to assess the anti-tumour efficacy of FTD-TPI drug in combination with oxaliplatin and an anti-mouse PD-1 monoclonal antibody (clone RMP1-14) using murine colorectal carcinoma (CRC)-bearing mice with survival parameters as endpoints. Anti-mouse PD-1 administration sequence has been tested to assess if sequence conditioned efficacy. The sequences tested on the in vivo study are sequential or concomitant and defined in mice model as described below and in FIG. 4. Concomitant schedule:

Administration of TAS-102 at Day 1 to Day 5 every week during 4 weeks;

Administration of Oxaliplatin at Days 2 (within 24 hours of administration of Tas-102 at Day 1), 9, 16 and 25;

Administration of the anti-mouse PD-1 antibody at Days 1, 3 and 5 every week. Sequential schedule:

Administration of TAS-102 at Day 1 to Day 5 every week during 4 weeks;

Administration of Oxaliplatin at Days 2 (within 24 hours of administration of Tas-102 at Day 1), 9, 16 and 25;

Administration of the anti-mouse PD-1 antibody at Days 8, 10 and 12, then every week.

One hundred and fifty (150) immune competent BALB/c mice have been injected with one million CT-26 cells re-suspended in 100 μl of RPMI (Roswell Park Memorial Institute medium) without FBS (foetal bovine serum). Once the tumours reach the target volume (50-70 mm), one hundred (100) out of one hundred fifty (150) has been randomized using Servier software into 5 groups (N=20/group) and the treatments has been administered during 4 weeks as shown on FIG. 4.

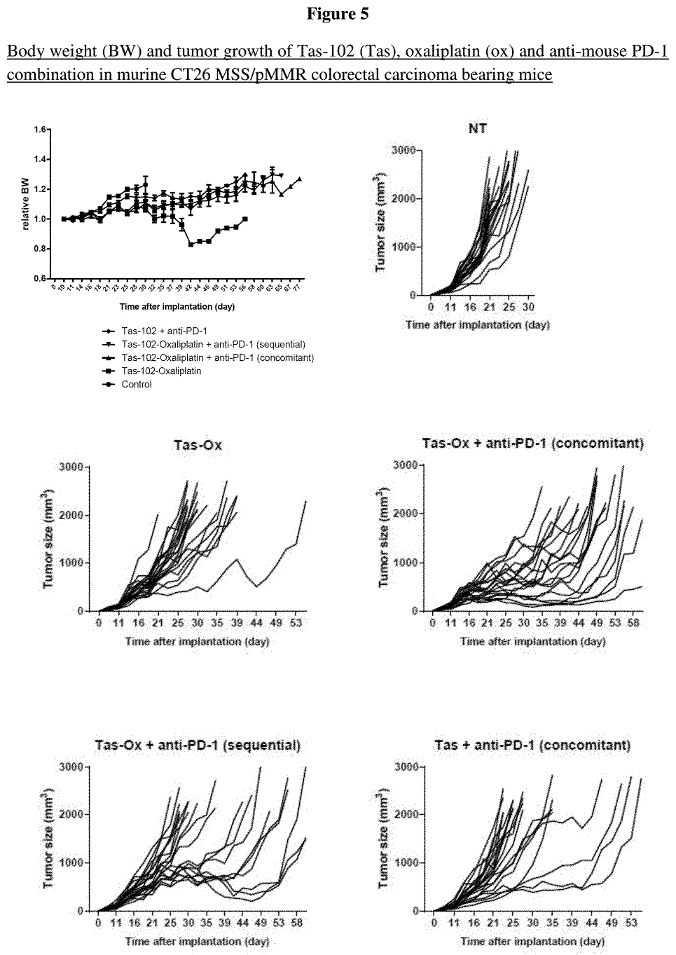

Tumor size and mice weight were monitored three times a week. Survival was assessed when tumor volumes were above 2500 mm³. In vivo, TAS-102+oxaliplatin, TAS-102+anti-PD-1, or TAS-102+oxaliplatin+anti-PD-1 exhibited a modest therapeutic effect on tumor growth, as shown on FIG. 5.

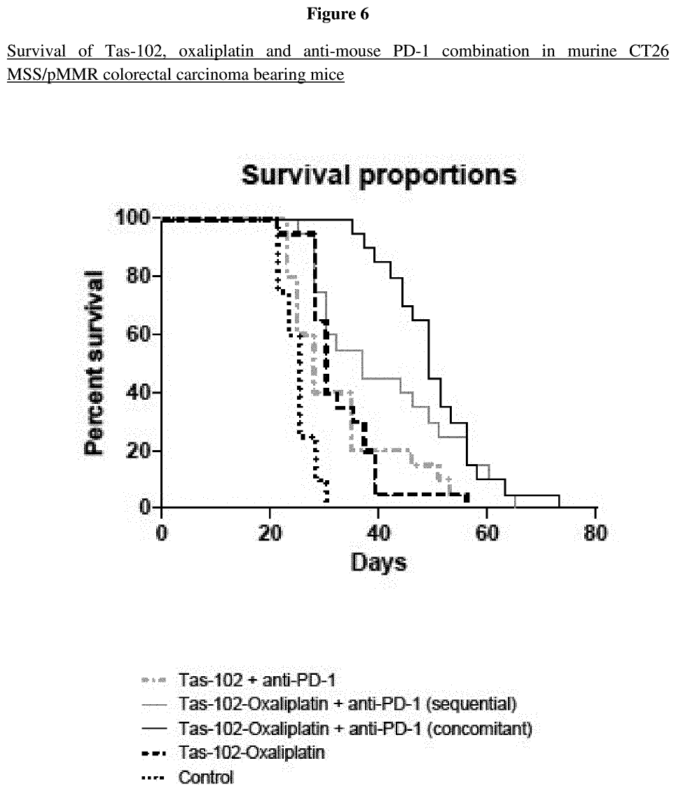

Notably, however, combination of FTD-TPI drug with oxaliplatin and anti-mouse PD-1 monoclonal antibody in sequential or concomitant administration during 4 weeks using murine CT26 MSS/pMMR colorectal carcinoma (CRC)-bearing mice showed higher statistical significant survival compared to FTD-TPI drug combined to anti-mouse PD-1 (p<0.05 and p<0.001 respectively) or the doublet chemotherapy FTD-TPI drug and oxaliplatin (p<0.02 and p<0.0001 respectively). See FIG. 6.

CLINICAL STUDY

A clinical Phase I dose-escalation study of TAS-102 in combination with oxaliplatin and nivolumab in metastatic colorectal cancer (mCRC) is planned. Triplet combination will be evaluated in a cohort of at least 35 evaluable patients receiving TAS-102, oxaliplatin and nivolumab as follows, on the basis of treatment cycle of 14 consecutive days:

TAS-102 will be administered orally bid (twice a day) at different doses (25 mg/m²/dose, 30 mg/m²/dose and 35 mg/m²/dose, depending on the dose level investigated and the tolerance) within 1 hour after completion of morning and evening meals, from Day 1 through Day 5. This will be followed by a recovery period of 9 days beginning on Day 6 through Day 14.

Oxaliplatin will be administered intravenously as 2-hours infusion at different doses (85 mg/m² or 65 mg/m² depending on the dose level investigated and the tolerance) on Day 1 of each treatment cycle. The start of infusion will be concomitant with the morning administration of TAS-102 at Day 1.

Nivolumab will be administered at a dose of 3 mg/kg, intravenously on Day 1 or after in a sequential administration at each treatment cycle.

Patients will be treated until progression of disease, unacceptable toxicity, investigator decision or patient refusal.

The study will be considered completed when all patients have discontinued from treatment or 12 months after the inclusion of the last patient whichever occurs first.

Tumour assessments will be performed throughout the study period and analysed using revised Response Evaluation Criteria in Solid Tumors (RECIST) criteria (Version 1.1, 2009). The date of disease progression and/or the date of death will be recorded for patients withdrawal from the study for a reason other than disease progression. The tumour assessments will be performed by Computed tomography scans (CT-scan):

at the baseline within 28 days before the first study drugs intake,

every 4 cycles, between D6-D14,

at the withdrawal visit at the investigator's discretion.

1-28. (canceled)

29. A combination comprising:
an FTD-TPI drug,
an anti-tumor platinum complex,
and an immune checkpoint modulator.

30. The combination according to claim 29, which is formulated for concomitant or sequential therapeutic use.

31. The combination according to claim 29, wherein the anti-tumor platinum complex is selected from cisplatin, carboplatin and oxaliplatin.

32. The combination according to claim 29, wherein the anti-tumor platinum complex is oxaliplatin.

33. The combination according to claim 29, wherein the immune checkpoint modulator is selected from an anti-PD-1 antibody or an anti-PD-L1 antibody.

34. The combination according to claim 29, wherein the immune checkpoint modulator is selected from nivolumab and pembrolizumab.

35. The combination according to claim 29, wherein the immune checkpoint modulator is nivolumab.

36. The combination according to claim 30, wherein each component is present at a dose that is 50 to 100% of the recommended dose for each component when administered alone.

37. The combination according to claim 30, which is formulated for concomitant administration of the three components.

38. The combination according to claim 30, which is formulated for sequential administration of the three components.

39. A method of treating cancer in a subject in need thereof, comprising administration of the combination according to claim 29, alone or in combination with one or more pharmaceutically acceptable excipients, wherein the components of the combination are administered concomitantly or sequentially.

40. The method according to claim 39, wherein administration of the immune checkpoint immunomodulator is initiated later than administration of the two other components.

41. The method according to claim 39, wherein the treatment cycle is 14 days.

42. The method according to claim 39, wherein the FTD-TPI drug is orally administered at a dose of 20 to 80 mg/m²/day in terms of FTD in two divided portions per day for five consecutive days, and then a 9-day rest period is taken.

43. The method according to claim 39, wherein the anti-tumor platinum complex is oxaliplatin and is administered at the dose of 60 to 90 mg/m² intravenously repeated every 2 weeks.

44. The method according to claim 39, wherein the immune checkpoint modulator is nivolumab and is administered at a dose of 3 mg/kg administered intravenously every 2 weeks.

45. The method according to claim 39, wherein the combination is administered during a 14 day treatment cycle wherein:
the FTD-TPI drug is administered orally bid (twice a day) at a dose of 25, 30 or 35 mg/m²/dose in terms of FTD from Day 1 to Day 5, followed by a recovery period of 9 days on Day 6 through Day 14;
the oxaliplatin is administered on Day 1 at 85 or 65 mg/m²; and
the nivolumab is administered at a dose of 3 mg/kg at Day 1 or at 24 hours to 14 days later.

46. The method according to claim 45, wherein:
the FTD-TPI drug is administered within 1 hour after completion of morning and evening meals;
the oxaliplatin is administered intravenously as a 2-hour infusion, the start of the infusion being concomitant with the morning administration of the FTD-TPI drug at Day 1; and
the nivolumab is administered intravenously, the start of the infusion being concomitant with the morning administration of the FTD-TPI drug at Day 1 or sequential 24 hours to 14 days later.

47. The method according to claim 46, wherein the nivolumab is administered at Day 1.

48. The method according to claim 46, wherein the nivolumab is administered 24 hours to 14 days after Day 1.

49. The method according to claim 41, wherein the 14 day treatment is repeated as long as the treatment combination will benefit to the patients.

50. The method according to claim 39, wherein the cancer is colorectal or gastric cancer.

51. The method according to claim 39, wherein the cancer is metastatic colorectal cancer.

52. A pharmaceutical composition comprising the combination according to claim 29, alone or in combination with one or more excipients.

53. The pharmaceutical composition according to claim 52, wherein the FTD-TPI drug, the anti-tumor platinum complex, and the immune checkpoint modulator are formulated for concomitant or sequential administration, and wherein each component is provided in effective amounts for the treatment of cancer.
